(19) United States
(12) Patent Application Publication
(10) Pub. No.: US 2024/0099685 A1
(43) Pub. Date: Mar. 28, 2024

(54) DETECTION OF DISEASES AND VIRUSES BY ULTRASONIC FREQUENCY

(71) Applicant: BLACK BOX VOICE LTD., Holon (IL)

(72) Inventors: Danny SIVAN, Yavne (IL); Ezra ORKOBY, Azor (IL)

(21) Appl. No.: 18/274,230

(22) PCT Filed: Jan. 28, 2022
(86) PCT No.: PCT/IB2022/050750

§ 371 Date: Jul. 26, 2023

Related U.S. Application Data
(60) Provisional application No. 63/142,522, filed on Jan. 28, 2021.

(51) Int. Cl. A61B 7/00; A61B 5/00; G10L 21/028; G10L 21/043; G10L 25/66

(52) U.S. Cl. CPC A61B 7/003; A61B 5/4803; A61B 5/6898; A61B 5/7267; G10L 21/028; G10L 21/043; G10L 25/66; A61B 2562/0204

(57) ABSTRACT

A method for detecting infection from a voice sample, the method including: generating machine learning (ML) training data, including: collecting raw data from a plurality of specimens, for each specimen: capturing an audio recording of internal sounds of the specimen inhaling and exhaling, capturing an audio recording of external sounds of the specimen inhaling and exhaling, and receiving medical data; training a ML model based on the training data; classifying a newly received audio recording of external sounds of a user, using the ML model; and outputting a metric determining a health status of the user.

### FIELD OF THE INVENTION

The present invention relates to method and systems for detecting disease and more specifically to detection of disease from a voice sample using machine learning.

### BACKGROUND OF THE INVENTION

Given the restrictions on mobility due to the global lock-down scenario resulting from the COVID epidemic, face-to-face medical consultations are difficult. However, the health industry continues to evolve and is adopting telemedicine to facilitate the accessibility of health services. Telemedicine is a blend of information and communication technologies with medical science. But telemedicine is limited by the apparent lack of physical examination, which in turn may increase the number of incorrect diagnoses. Therefore, a physical examination seems to be mandatory process for proper diagnosis in many situations. For example, every doctor has a stethoscope, but how many people own a personal stethoscope? Digital stethoscopes currently on the market usually do not pay off on a personal level, even in developed countries.

### SUMMARY OF THE INVENTION

There is provided a solution that combines any standard stethoscope with a microphone having sufficient bandwidth for recording the sound of the heart and/or the respiratory system, for example via a smartphone. A designed application that identifies the device being used records the auscultation for signs of specific symptoms and introduce these voices to an AI/machine learning algorithm. This project is currently in conjunction with potential symptoms of upper respiratory tract infection, chronic obstructive pulmonary disease, or pneumonia, as these are the most common symptoms associated with COVID-19.

The same AI model can be deployed on different devices and the core BBV process remains the same. Indeed, the BBV application can run on a personal device, such as a microcontroller, a mobile phone, or even on a personal computer (PC).

According to the present invention there is provided a method for detecting infection from a voice sample, the method including: (a) generating machine learning (ML) training data, including: (i) collecting raw data from a plurality of specimens, for each specimen: capturing an audio recording of internal sounds of the specimen inhaling and exhaling, capturing an audio recording of external sounds of the specimen inhaling and exhaling, and receiving medical data, such that the training data includes: (A) an internal dataset of a plurality of the audio recordings of internal sounds of a plurality of specimens inhaling and exhaling, (B) an external dataset of a plurality of the audio recordings of external sounds of the plurality of specimens inhaling and exhaling, and (C) a medical dataset of medical information related to each of the specimens; (ii) processing the internal and external datasets to generate processed data and metrics for each of the internal and external datasets; (iii) correlating between the internal dataset, the external dataset and the medical dataset; (b) training a ML model based on the training data; (c) classifying a newly received audio recording of external sounds of a user, using the ML model; and (d) outputting a metric determining a health status of the user.

According to further features the audio recording of internal sounds and the audio recording of external sounds are synchronized. According to further features the audio recording of internal sounds and the audio recording of external sounds are unsynchronized. According to further features each audio recording of internal sounds is captured by a specialized recording device approximating auscultation of a thorax. According to further features each audio recording of internal sounds is captured by pressing an audio recorder against a thorax of the specimen. According to further features each audio recording of external sounds is captured by a commercial recording device. According to further features each audio recording of external sounds is captured by a recording device held away from a face of the specimen.

According to further features the specimen inhaling and exhaling is achieved by the specimen performing at least one action selected from the group including: coughing, counting, reciting a given sequence of words.

According to further features the processing includes: bandpass filtering of raw data of the internal dataset and the external dataset to produce a bandpass filtered data set.

According to further features processing includes: detecting a rhythm in each of the plurality of audio recordings of external sounds or the plurality of audio recordings of the internal sounds. According to further features the rhythm is compared to a reference rhythm having an associated reference tempo, and a data set tempo generated for the external dataset or the internal dataset, the data set tempo being in reference to the associated reference tempo. According to further features the method further includes: adjusting the data set tempo to match the reference tempo, thereby producing a prepared data set and a corresponding tempo adjustment metric.

According to further features the method further includes: detecting and removing spoken portions of the prepared data set to produce a voice-interims data set.

### BRIEF DESCRIPTION OF THE DRAWINGS

Various embodiments are herein described, by way of example only, with reference to the accompanying drawings, wherein:

FIG. 1 is an exemplary band pass filter circuit;

FIG. 2 is a flow chart 200 of the instant process;

FIG. 3 is a picture of a thorax and indication of the position of the stethoscope for proper data collection;

FIG. 4 includes a number of screenshots from an example user interface;

FIG. 5 is an example screen of the user interface;

FIG. 6 is an example screen depicting the NN Classifier user interface;

FIG. 7 is an example output screen of the user interface;

FIGS. 8A-8F various app screens of the mobile app.

### DESCRIPTION OF THE PREFERRED EMBODIMENTS

The principles and operation of learning model and diagnostic methodology according to the present invention may be better understood with reference to the drawings and the accompanying description.

It is noted that throughout this document the terms Artificial Intelligence (AI), Machine Learning (ML), Neural Network (NN), Deep Learning and similar terms are used interchangeably and only for the purpose of example. The term Machine Learning (ML) will be used herein as a catchall phrase to indicate any type of process, algorithm, system and/or methodology that pertains to machine learning, such as, but not limited to, AL, ML, NN, Deep Learning and the like.

### Hardware

Audio Processing is an integral part of the instant system. The instant systems and methods deal with biomedical signals. Accordingly, it is necessary to ensure that only the data of interest is examined from the signal and everything else is filtered out.

In subjects with healthy lungs, the frequency range of the vesicular breathing sounds extends to 1,000 Hz, and the majority of power within this range is found between 60 Hz and 600 Hz. Other sounds, such as wheezing or stridor, can sometimes appear at frequencies above 2,000 Hz. In the range of lower frequencies (<100 Hz), heart and muscle sounds overlap. This range of lower frequencies (<100 Hz) is preferably filtered out for the assessment of lung sounds.

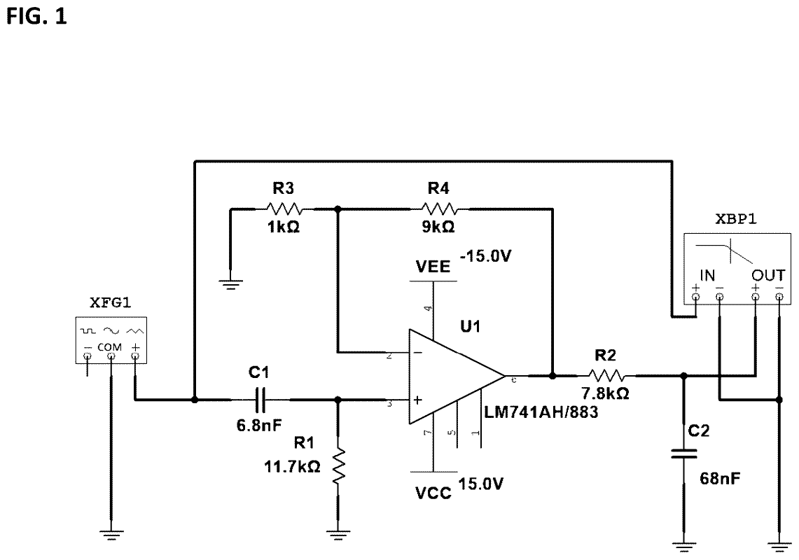

Hence, to reduce the influence of heart and muscle sounds, as well as noise, and to prevent aliasing (misidentifying a signal frequency, introducing distortion or error), all sound signals are band pass-filtered, using a band pass of 100 Hz to 2,100 Hz. FIG. 1 illustrates an exemplary band pass filter circuit. The band pass filter circuit substantially eliminates sounds other than from the lungs. Implementations can be done without connecting a filter circuit, even though this normally results in loss of some accuracy when subsequently detecting abnormal breathing/voice sounds using the model.

### Training ML Model

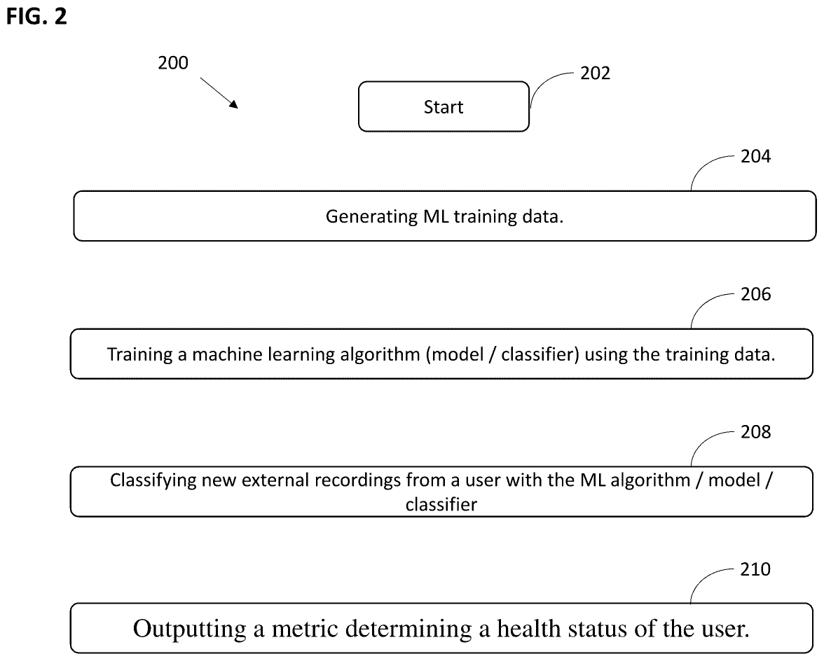

An exemplary implementation of using the instant system is now described. Implementations include methods for determining a state of a person's body, in particular the person's health status, detection of disease, illness, and/or condition of the body. FIG. 2 depicts a flow chart 200 of the instant process. The method starts at step 202 and includes:
a. Step 204: Generating neural network training data. Typically, two datasets are used. More than two datasets can also be used, with the described method adjusted accordingly.

1. A first dataset is provided, typically as raw data of audio recordings from the person's chest area (thorax) while the person is inhaling and exhaling (speaking, coughing, breathing). The term “person”, as used herein to denote the individual from whom the raw audio data is attained, may also be referred by the terms “subject”, “specimen”, “participant”, “sample source”, variations thereof and similar phrases. These terms or phrases are used interchangeably herein.

The first dataset is referred to in this document as the “true”, “actual”, “inside” or “internal” data. For best results, the first dataset (also referred to herein as “internal dataset”) should be recorded as accurately as possible, using a high-quality device. One such device is a digital stethoscope. As is known in the field, “auscultation” is the medical term for using a stethoscope to listen to the sounds inside of a body. For the current method, auscultation of the lungs is preferred. In examples, each audio recording of internal sounds is captured by a specialized recording device approximating auscultation of a thorax. In examples, each audio recording of internal sounds is captured by pressing an audio recorder against a thorax of the specimen

2. A second dataset is provided, typically as raw data of a person inhaling and exhaling, similar to the first dataset. The second dataset (also referred to herein as the external dataset) is referred to in the context of this document as “environmental”, “measured”, “outside”, or “external” data, and is provided using a commercial microphone (such as a built-in smartphone or personal computer (PC) microphone). Each audio recording of external sounds is captured by a recording device held away from the subject's face. Preferably the audio in the two datasets (first and second datasets) are the same, for example, the person coughing, counting, or speaking a given sentence or reciting a given sequence of words. Preferably, the first and second datasets are captured at the same time, for best correlation, for example, microphones synchronized in time. However, this is not limiting, and the recordings of the first and second datasets can be unsynchronized recordings of the same audio (same reading or noise like coughing).

3. A medical dataset of medical information related to each person for whom internal and external recordings are provided. The medical information may include medical diagnosis of the health status of the person. For example, whether the person is healthy or ill, what diseases (if any) the person has, and/or what is the state of the person's health. Diseases include, but are not limited to: Covid-19, flu, cold, Chronic Obstructive Pulmonary Disease (COPD), Pneumonia, and cancer. Health status may also include pregnancy and alcohol consumption. In the context of this document, for simplicity of description, the terms “medical information”, “disease” and “health status” may be used interchangeably for diseases, health status, and other status (such as gender).

4. For each of the first and second (internal and external) datasets various layers of processing can be done to generate a variety of processed data and metrics. Some exemplary layers of processing include the following:
i. Bandpass filtering the raw data to produce a bandpass filtered data set. See the above description. Typically, bandpass filtering in the range of 100 Hz to 2100 Hz is sufficient. Other ranges can be used, depending on the application and diseases to be identified (or ignored).
ii. Detecting the rhythm. For example, an audio data set (recording) of coughing will have a rhythm of a strong, regular, repeated pattern of sound for each cough. Similarly, an audio data set of counting will have a rhythm for each number, or a spoken sentence may have a typical cadence. Once the rhythm is detected, the rhythm can be compared to a reference rhythm having an associated reference tempo, and a data set tempo generated for the data set, the data set tempo in reference to the reference tempo.
iii. Adjusting the data set tempo to match the reference tempo, thereby producing a prepared data set and corresponding tempo adjustment metric. Each data set may be adjusted so the tempo is quickened/condensed or slowed/expanded. The tempo adjustment metric represents the increase/decrease of tempo of the data set to match the reference tempo. Tempo can also be thought of as the “pace” of the spoken audio, with an index being a metric of the adjustment to a reference beat. The original tempo can also be used as a metric.
iv. Detecting and removing spoken portions of the prepared data set to produce a voice-interims data set. The voice-interims data set can at first be thought of as a collection of the “silence” between the spoken audio. However, this “silence” is actually non-spoken audio, or other body sounds that occur between the spoken audio. The voice-interims can include sounds from before the intended parts of speech, such as rasping, exhalation, and/or vibrations. Similarly, the voice-interims can include sounds after the intended spoken audio, such as further exhalation, rasping, etc. Inhalation or exhalation between the intended audio can be included in the voice-interims. In the context of this document, voice-interims are between the person's intended exhalation of audio, thus sounds like coughing are included in the intended audio.

5. Once pre-processing has been completed, a variety of processed data and metrics has been generated, for each data set, and corresponding between the first and second datasets. Next, correlating is done between the first and second datasets of the processed data and metrics to generate training data. A typical correlation includes, the prepared first data set, the first tempo adjustment metric, the first voice-interim data set, the prepared second data set, the second tempo adjustment metric, the second voice-interim data set, and the health status/medical information. The training data preferably includes the results of the correlating step, as well as the data used to do the correlation. The training data may include other data, whether or not used to do the correlation.
b. Once training data has been generated, at step 206, an artificial neural network (ANN) can be trained using the training data to generate a classifier (also known in the field as a model or ML model). The classifier has inputs including the prepared second data set (consumer grade recording from the user), the second tempo adjustment metric, and the second voice-interim data set. the classifier has outputs including metrics (for example, percentages) indicating how well the audio recording of the person matches a given set of health conditions (health statuses/medical information).
c. Once the classifier has been generated, the classifier can be used to classify user data (second data sets) at step 208. For example, a raw second data set (consumer recording) is received from a user. This second data set is typically recorded using commercial grade microphone, however this is not limiting, and if a higher quality microphone (such as a digital stethoscope) is available, the higher quality recording can be used. In either case, the data from the user to be processed is referred to as the “second data set”. The user second data set is pre-processed according to the above steps [(4)(i) to (4)(iv)], similar to how the data is processed for training to produce classifier inputs, but without correlation (as only one data set is needed to evaluate the health status of the user).
d. The (pre-)processed prepared data, raw data, and metrics (such as the tempo adjustment metric and voice-interim data set) are input to the classifier, and the classifier generates output metrics, at step 210, determining the person's health status.

The output metrics from the classifier may be post-processed as appropriate to generate more meaningful, or alternative representations of the person's health status.

### The Software and the Flow

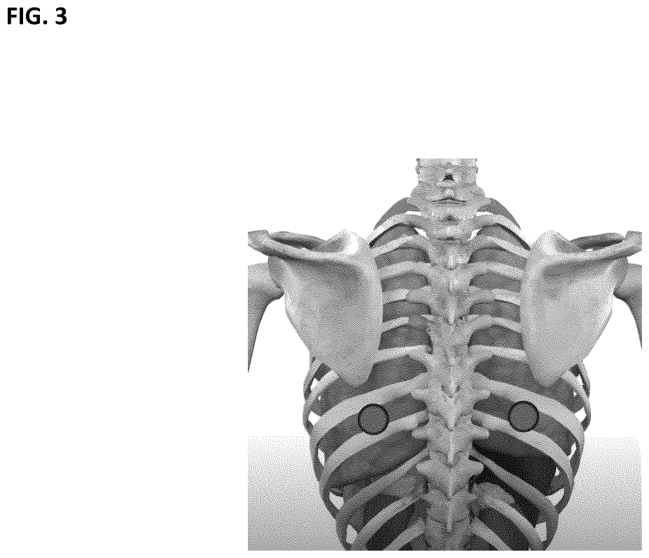

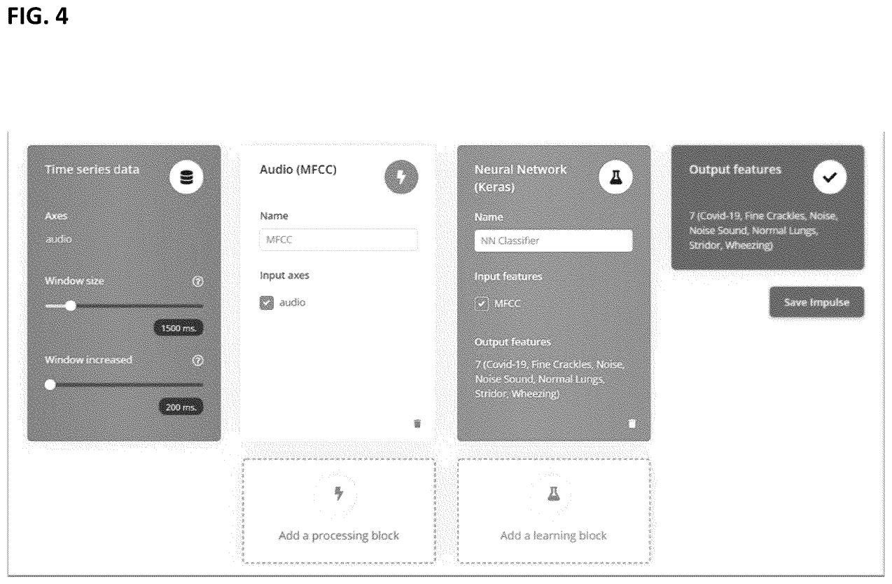

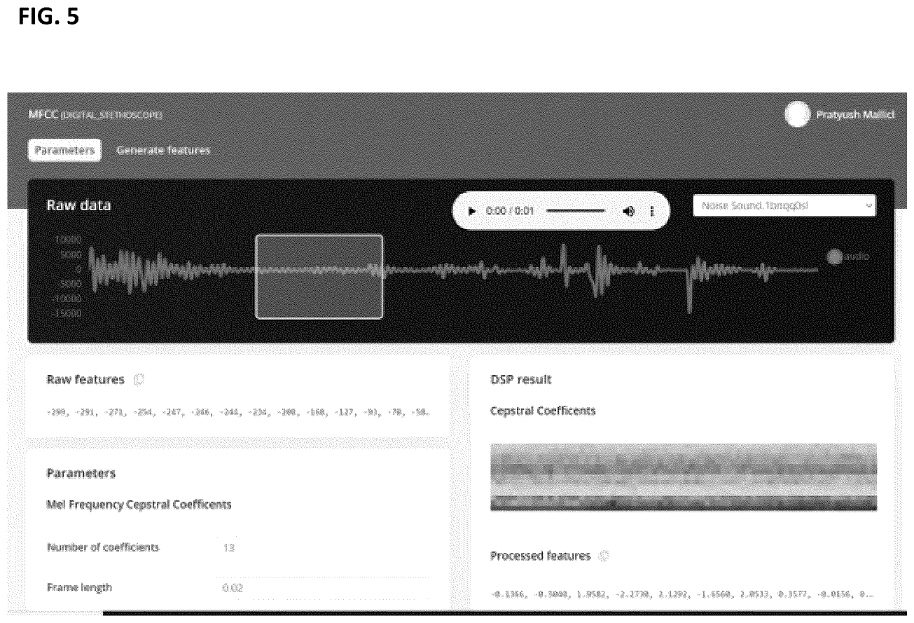

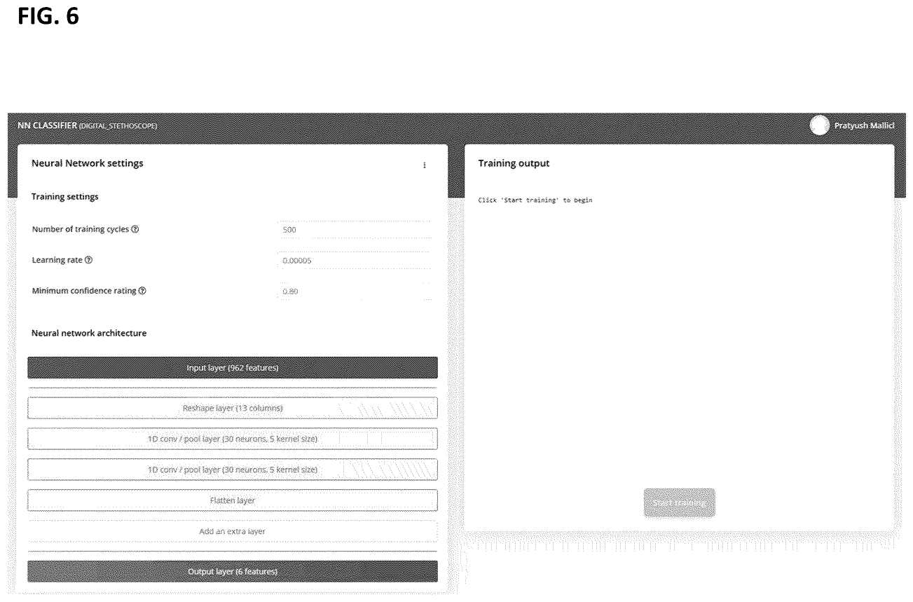

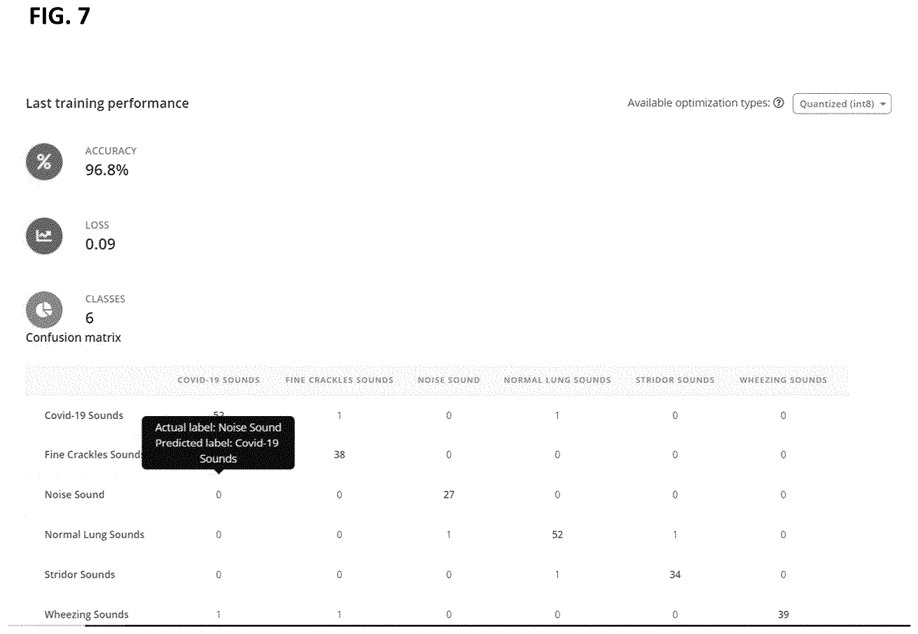

For completeness, a run-through of using an example implementation of the system in a software application is detailed hereafter.
1. To get the current exemplary implementation of the software up and running a user has to first sign up for a service account (hereafter also “account”).
2. After entering information and verifying email, the user will be greeted by a “Let's get started” page. This will walk the user through the process of connecting a device, gathering data, and finally deploying a model.
3. Next, the user connects a device to the account. The device can be anything from a microcontroller to a phone or a laptop.
4. The model would be trained on the data acquired by various connected devices, hence the model will give best results when identifying the type of device sending input.
5. Next, go to data collection tab in the system site, and connect the stethoscope-microphone setup to a local PC (used here as an example, or smart phone, or other sound collection device). Tuning the PC microphone setting allows for most accurate results.
6. After this, in the data collection tab, the user selects Options under “Record new data” label; chooses microphone as the sensor with the highest sampling rate as to prevent losing any important signals; names the type of sound in which to record in the “Label” option; and selects the data acquisition device to record the samples. A sample could be of any length as long as it contains enough data to generate features. The standard is set at 10 seconds.
7. The user clicks on the RAW DATA tab to begin sampling. The user will be prompted to allow access to record data from the device. Once the access has been granted—the sampling begins. FIG. 3 depicts a picture of a thorax and indication of the position of the stethoscope for proper data collection. The digital stethoscope, or other recording device, should be pressed against the chest of on the subject to best record internal sounds.
8. The user is directed to inhale and exhale (e.g., cough, count, talk, etc.) for the selected time period. The data is then uploaded. Once the data has been uploaded, a new line will appear under ‘Collected data’. The waveform of the audio will also appear in the ‘RAW DATA’ box. The user can use the controls underneath to listen to the audio that was captured. The user may repeat this process until satisfied with the variants of different labels of data from the sample. It may take around one minute i.e., 6×10 seconds per sample of data for each of the different categories of sound provided for the model to detect.
9. After data acquisition is done successfully, the data has to be created to use to produce the model/classifier and define the dataset's parameters. The instant system provides a simplified process and interface for integrating the captured data. FIG. 4 includes a number of screenshots from an example user interface. With the training dataset in place, an impulse can be designed. An impulse takes the raw data, slices the raw data up in smaller windows, uses signal processing blocks to extract features, and then uses a learning block (neural network [NN] classifier, model) to classify new data. Signal processing blocks always return the same values for the same input and are used to make raw data easier to process, while learning blocks (the NN classifier is) are able to learn from past experiences.
10. According to an example implementation, an “MFCC” signal processing block is provided. MFCC stands for Mel Frequency Cepstral Coefficients. The signal processing turns raw audio (which contains a large amount of redundant information) into a simplified form. The simplified audio data is then passed into a Neural Network (NN) block (classifier), which is/will be trained to distinguish between the various classes of audio. Since this model is mostly used on phones or laptops, memory is not a constraint, allowing to train as many classes (diseases, states of a person's body, etc.) as determined necessary or desirable.
11. The system algorithm slices up the raw samples into windows that are fed into the machine learning model during training. The Window size field controls how long, in milliseconds, each window of data should be. A one-second audio sample will be enough to determine unwanted background noise, such as whether a faucet is running or not, so the Window size is preferably set to 1000 ms. Using the interface, one can either drag the slider or type a new value directly. Each raw sample is sliced into multiple windows, and the Window increase field controls the offset of each window.
12. Subsequent windows are derived from the first determination. For example, a window size value of 1000 ms would result in each window starting 1 second after the start of the previous one.
13. By setting a ‘Window increased’ size that is smaller than the ‘Window size’, a user can create windows that overlap. Although they may contain similar data, each overlapping window is still a unique example of audio that represents the sample's label. By using overlapping windows, the training data is more fully taken advantage of. For example, with a Window Size of 1000 ms and a Window Increase of 200 ms, the system can extract 10 unique windows from only 2 seconds of data.
   The interface in FIG. 4 depicts screens and clickable buttons for setting up an impulse. It is made clear that the interface design is merely an example, and not intended to limit the scope of the system and method in any way. To set up an impulse, The ‘Window Size’ and ‘Window increase’ are set, for example, as described above. Next, the ‘Add a processing block’ icon is clicked and the user can select the type of processor. In the example, the ‘MFCC’ block is chosen. Next, the user clicks on the ‘Add a learning block’ icon and selects ‘Neural Network (Keras)’ block. Finally, the ‘Save Impulse’ button is clicked.
14. After assembling the building blocks of the Impulse, the user can configure each individual part. The interface provides one or more screens for further configuration. An example screen is depicted in FIG. 5. The system can be presented as a service with a website or mobile application interface. Hereafter the system and the interface may be interchangeably used. The interface may be referred to herein as “service”, “site”, “website” and “app”. The Service provides sensible defaults that will work well for many use cases. As such, in most cases, the default values can be left unchanged.
15. The data from the MFCC is passed into a neural network architecture that is good at recognizing patterns in tabular form of data. Before training the neural network classifier, the features must be generated. This can be achieved by clicking the ‘Generate features’ button at the top of the page, and then clicking the green ‘Generate features’ button that is presented on the screen (not shown). This is the last step in the preprocessing, prior to training the NN classifier.
16. With all the preprocessing the user can now ready to train the neural network with this data. FIG. 6 is an example screen depicting the NN Classifier user interface. It is suggested to proceed with the default settings that have been generated for the model. Once the first training is completed the user can tweak these parameters to make the model perform accurately. To begin the training, the ‘Start training’ button is clicked. Training will take a few minutes.
17. FIG. 7 depicts an example output screen. In the example, a ‘Last training performance’ panel is depicted. After the initial train cycle has run its course and the ‘Last training performance’ panel has been displayed, the user can change some values in the configuration. The ‘number of training cycles’ refers to the parameter relating to how many times the full set of data will be run through the neural network during training. In the example, the number is set to 500.

If too few cycles are run, the network may not manage to learn everything it can from the training data. However, if too many cycles are run, the network may start to memorize the training data and will no longer perform well on data it has not seen before. This is called overfitting. As such, the aim is to get maximum accuracy by tweaking the parameters.

The ‘minimum confidence rating’ refers to the threshold at or below which a sample will be disregarded. For example, a setting of 0.8 means that when the neural network makes a prediction, and there is a 0.8 probability that some audio contains a noise, the machine learning (ML) algorithm will disregard it, unless it is above the threshold of 0.8.
18. Even though the model shows good accuracy, it is important to test it out on real data. The system includes interface keys for starting a live classification. The interface screens (not shown) allow for selecting the capture device and controls for stating and stopping the sampling process. For example, the user can capture 5 seconds of background noise. The sample will be captured, uploaded, and classified. Once this has happened, a breakdown of the results will be shown.

### Detection of Asymtomatic Covid-19 Infections Through Device-Recorded Coughs

People without symptoms infected with Covid-19, or other diseases show, by definition, no noticeable physical symptoms of the disease. Thus, they are less likely to perform for virus tests and can spread the infection, without their knowledge. But asymptomatic people are not completely free of changes caused by the virus. Testing has found that asymptomatic people sound different from healthy people. These differences cannot be deciphered to the human ear, but these differences can be collected by artificial intelligence. An AI model, according to the instant disclosure, as able to differentiate between asymptomatic and healthy people through forced cough recordings, which people transmit via Internet web browsers or dedicated applications, using devices such as PCs, laptops, tablets, cell phones and other devices.

Applicants trained the model on hundreds of cough samples as well as spoken words. When they fed the model in new cough recordings, the model accurately identified 98.5 percent of the coughs from people who were confirmed to have Covid 19, including 100 percent of the coughs from asymptomatic patients—who reported having no symptoms but tested positive for the virus.

A user-friendly mobile application (hereafter “testing app”) is provided as a non-invasive pre-screening tool to identify people who may be symptomatic or asymptomatic for Covid-19. For example, a user can log in daily, cough on their phone and get immediate information on whether s/he may be infected. When a positive result is received, the app user may be directed confirm with a formal examination, such as a PCR test. In some implementations, a formal test is not required due to the proven accuracy of the system.

A biomarker is a factor objectively measured and evaluated which represents a biological or pathogenic process, or a pharmacological response to a therapeutic intervention, which can be used as a surrogate marker of a clinical endpoint [19]. A vocal biomarker is a signature, a feature, or a combination of features from the audio signal of the voice and/or cough that is associated with a clinical outcome and can be used to monitor patients, diagnose a condition, or grade the severity or the stages of a disease or for drug development. It must have all the properties of a traditional biomarker, which are validated analytically, qualified using an evidentiary assessment, and utilized.

According to embodiments there is provided a Black Box Voice (BBV) App (also referred to as “testing app”) which is a software app which analyzes vocal biomarkers and uses Artificial Intelligence (AI) as a medical screening tool. The software application can detect COVID-19, using a combination of unique vocal samples and a trained AI model and provide a positive/negative indication within minutes of the sampling operation, all on a common mobile smartphone.

The testing app can distinguish symptomatic, as well as asymptomatic COVID-19 patients from healthy individuals. The coronavirus symptoms (even in asymptomatic patients) initially causes infection in the areas of the nasal cavity and throat and then infects the lungs. Therefore, the voice-affecting parts of the body are the nasal passages, the throat and the lungs. These changes can be detected at any stage of the COVID-19 infected patient. Based on these vocal changes, there is a distinct vocal biomarker consisting of a combination of features from the audio signal of the acquired voice and cough signals that is associated with a clinical outcome and can be used to diagnose COVID-19.

The interactive app instructs the patient to count to three and then cough three times. The smartphone microphone captures the voice and cough samples and converts the audio signals into “features”, meaning the most dominating and discriminating characteristics of the signal, which comprise the detection algorithm. These “features” include, prosodic features (e.g., energy), spectral characteristics (e.g., centroid, bandwidth, contrast, and roll-off), voice quality (e.g., zero crossing rate) as well as other and methods of analysis including Mel-Frequency Cepstrum Coefficient (MFCCs), Mel Spectrogram, etc.

The BBV App algorithm located at the backend, consisting of the selection of “features”, automatically classifies the incoming data according to the appropriate clinical outcome (i.e., positive or negative for COVID-19). The results are presented to the user on the smartphone screen within 60 seconds.

### Using the App:

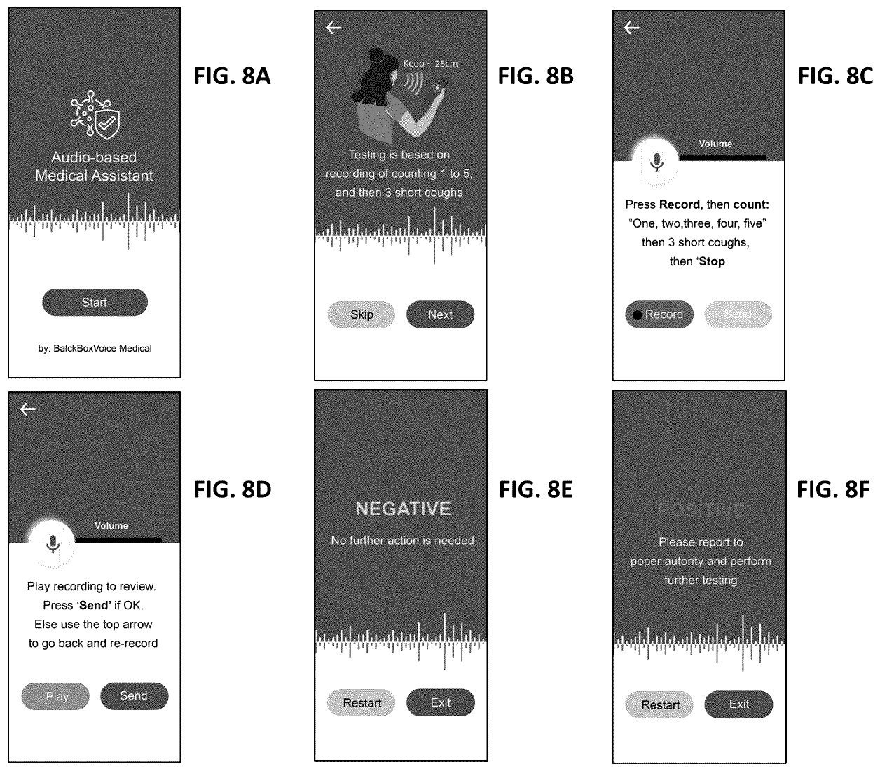

Once the app is installed, a test may be started according to the following steps, depicted in app screens in FIGS. 8A-8F.

Start the app by clicking the Start button on the screen in FIG. 8A. Next, a screen depicted in FIG. 8B includes a pictorial and written explanation on the required recording content and analysis. The picture shows that the recording device should be held about 25 cm away from the mouth. The instructions explain that the user will need count from 1 to 5 and then cough three times while recording. FIG. 8C depicts a recording screen. Pressing the red ‘Record’ button starts the recording. Once pressed, the screen changes to “Recording” and displays the instructions to count and cough again and then to press the ‘Stop’ button. Once done, the screen depicted in FIG. 8D appears. The recording may be reviewed by pressing the ‘Play’ button. The recording is sent for analysis by pressing the ‘Send’ button. Following a short wait, the test results are displayed. A NEGATIVE result is displayed in GREEN (FIG. 8E), while a POSITIVE result is displayed in RED (FIG. 8F).

Validation of the BBV App device software was performed according to the IEC 62304:2006/AMD1:2015 standard for Medical device and Software life-cycle processes standards, including usability engineering to medical devices. The software related documents were composed according to the specific IEEE standards and the FDA Guidance for the Content of Premarket Submissions for Software Contained in Medical Devices. The BBV App device software is finalized and frozen prior to pursuing the current clinical study. The following software validation documents are maintained on file at the company as part of the Design History File:
Software Development and Lifecycle Procedure (Software Development Environment Description) (Doc. No. QSR-0401-00)
Risk/Hazard Analysis (Doc. No. RMF-0001-00)
Software Requirements Specifications (SRS) (Doc. No. SRS-0001-00)
Software Detailed Design (SDD) (Doc. No. SDD-0001-00)
Software Test Description (STD) (Doc. No. STD-0001-00)
Software Test Report (STR) (Doc. No. STR-0001-00)
Software Version Description (SVD)
Software Traceability Matrix (appears in SRS, SDD and STD)
Software Version Description (Doc. No. SVD-0001-00)

### Clinical Pilot Study Validation

Following the above described algorithm development and validation, the algorithm parameters of the BBV App to detect COVID-19 were finalized. A pilot clinical study, consisting of 546 subjects, was performed to further validate the BBV App device algorithm in the clinical environment in which it is intended to be used. The pilot clinical study is described here.

A preliminary study was conducted to compare the BBV App device results obtained from voice recordings for the non-invasive detection of COVID-19, using invasive nasal swab specimens and PCR analysis findings as the gold, reference standard. Active enrollment took place from the March 2021 to July 2021 at Ashdod Rashbi Medical Center, Maccabi Health Care Services (Ashdod, Israel, Dr. Gil Siegal-PI) and Al Mazroui Medical Center (Dubai, United Arab Emirates, Dr. Vinash Kamal-PI). A total of 546 eligible subjects were enrolled in the study. The study population, who represent the target population for this procedure, consisted of healthy subjects and subjects with known or suspected COVID-19 disease, who were scheduled for invasive nasal swab tests, which were subsequently analyzed using the polymerase chain reaction (PCR) test method.

Subjects of both genders, >18 years of age were recruited to the study.

Nasal swab specimen acquisition was performed in a routine fashion in healthy subjects and in subjects with suspected COVID-19 disease. PCR testing of each nasal swab specimen was analyzed and served as the gold standard reference. The BBV Medical App results were not used for diagnostic or clinical decisions. The blinding status was maintained until the last subject completed the study at each site.

The dichotomous determination (positive or negative) of the BBV App device result per patient was compared to the PCR result for the same patient. The sensitivity and specificity of the BBV App device was calculated. Furthermore, the BBV App device accuracy, positive predictive value and negative predictive value were determined.

A total of 546 subjects were analyzed in the main analysis set. The data represents the general patient population undergoing testing for COVID-19 and in whom the BBV App device may potentially be used.

In 403/546 (73.8%) of the subjects, the PCR results confirmed a negative finding for COVID-19 and in 143/546 (26.2%) of the subjects the PCR results indicated a positive finding for COVID-19. The BBV App device indicated a negative finding in 406/546 (74.4%) of the subjects and indicated a positive finding in 140/546 (25.6%) of the subjects. Statistical analysis of the 546 study subjects presented results for the study primary endpoints of sensitivity and specificity of 97.9% and 100%, respectively exceeding the goal of the primary objective. The lower limit of the 95% confidence interval demonstrates the successful achievement of the primary objective goals, sensitivity and specificity as well, at 95.6% and 100% respectively. The Exact binomial P-value's (1-sided) were <0.001, respectively, deeming the results in 546 subjects statistically significant.

The first secondary endpoint presented 99.5% accuracy in correctly measuring a positive or negative result. The second and third secondary endpoints, Positive Predictive Value (PPV) (100%) and Negative Predictive Value (NPV) (99%), further demonstrate the successful achievement of the secondary objectives of the study.

Thus, the study primary and secondary endpoints have been demonstrated as successfully met and support the safety and efficacy of the BBV App device for its intended use of detecting COVID-19 using voice recordings.

The clinically and statistically significance results of the BBV App device, demonstrate an effective screening device effective for providing an accurate and clinically meaningful, COVID-19 result using non-invasive voice recordings. The use of the BBV App device as a screening tool is an effective means of detecting COVID-19 infection, with additional caveats for interpreting positive and negative results (as stated in the device description section below) and can assist the physician in quickly determining treatment options. The results of the above clinical study will be corroborated in the current clinical study, in which usability in the hands of potential end users will also be assessed.

### Stage 2 Clinical Study Validation

Following the pilot validation clinical study consisting of 546 subjects, the stage 2 validation clinical study will be conducted and is described in the study protocol attached to this Helsinki Submission. The Stage 2 validation clinical study will be conducted according to the MOH—Department of Laboratories—Guidelines for Validation of Point of Care Testing (POCT) for detecting the SARS-CoV-2 Virus (18 Nov. 2020).

While the invention has been described with respect to a limited number of embodiments, it will be appreciated that many variations, modifications and other applications of the invention may be made. Therefore, the claimed invention as recited in the claims that follow is not limited to the embodiments described herein.

What is claimed is:

1. A method for detecting infection from a voice sample, the method comprising:
(a) generating machine learning (ML) training data, including:
(i) collecting raw data from a plurality of specimens, for each specimen: capturing an audio recording of internal sounds of said specimen inhaling and exhaling, capturing an audio recording of external sounds of said specimen inhaling and exhaling, and receiving medical data, such that said training data includes:
(A) an internal dataset of a plurality of said audio recordings of internal sounds of a plurality of specimens inhaling and exhaling,
(B) an external dataset of a plurality of said audio recordings of external sounds of said plurality of specimens inhaling and exhaling, and
(C) a medical dataset of medical information related to each of said specimens;
(ii) processing said internal and external datasets to generate processed data and metrics for each of said internal and external datasets;
(iii) correlating between said internal dataset, said external dataset and said medical dataset;
(b) training a ML model based on said training data;
(c) classifying a newly received audio recording of external sounds of a user, using said ML model; and
(d) outputting a metric determining a health status of said user.

2. The method of claim 1, wherein said audio recording of internal sounds and said audio recording of external sounds are synchronized.

3. The method of claim 1, wherein said audio recording of internal sounds and said audio recording of external sounds are unsynchronized.

4. The method of claim 1, wherein each said audio recording of internal sounds is captured by a specialized recording device approximating auscultation of a thorax.

5. The method of claim 1, wherein each said audio recording of internal sounds is captured by pressing an audio recorder against a thorax of said specimen.

6. The method of claim 1, wherein each said audio recording of external sounds is captured by a commercial recording device.

7. The method of claim 1, wherein each said audio recording of external sounds is captured by a recording device held away from a face of said specimen.

8. The method of claim 1, wherein said specimen inhaling and exhaling is achieved by said specimen performing at least one action selected from the group including: coughing, counting, reciting a given sequence of words.

9. The method of claim 1, wherein said processing includes:
bandpass filtering of raw data of said internal dataset and said external dataset to produce a bandpass filtered data set.

10. The method of claim 1, wherein said processing includes:
detecting a rhythm in each of said plurality of audio recordings of external sounds or said plurality of audio recordings of said internal sounds.

11. The method of claim 10, wherein said rhythm is compared to a reference rhythm having an associated reference tempo, and a data set tempo generated for said external dataset or said internal dataset, said data set tempo being in reference to said associated reference tempo.

12. The method of claim 11, further comprising:
adjusting said data set tempo to match said reference tempo, thereby producing a prepared data set and a corresponding tempo adjustment metric.

13. The method of claim 12, further comprising:
detecting and removing spoken portions of said prepared data set to produce a voice-interims data set.
